(19) United States
(12) Patent Application Publication
(10) Pub. No.: US 2024/0374115 A1
(43) Pub. Date: Nov. 14, 2024

(54) ENDOSCOPE SYSTEM, CONTROL METHOD, AND RECORDING MEDIUM

(71) Applicant: OLYMPUS CORPORATION, Tokyo (JP)

(72) Inventors: Masafumi HARAGUCHI, Tokyo (JP); Naoya HATAKEYAMA, Tokyo (JP)

(73) Assignee: OLYMPUS CORPORATION, Tokyo (JP)

(21) Appl. No.: 18/776,375

(22) Filed: Jul. 18, 2024

Related U.S. Application Data
(63) Continuation of application No. PCT/JP2022/003245, filed on Jan. 28, 2022.

(51) Int. Cl. A61B 1/00

(52) U.S. Cl. CPC A61B 1/00009; A61B 1/00006; A61B 1/00045

(57) ABSTRACT

An endoscope system includes: an endoscope acquiring a captured image; and a processor configured to: detect a region of interest in the captured image; select a portion of the captured image as a display area and generate an observation image from the display area; and cooperatively execute digital tracking processing and physical tracking processing. The digital tracking processing is processing in which a first target region in the display area is made to track the region of interest by changing a position of the display area in the captured image. The physical tracking processing is processing in which a second target region in the captured image is made to track the region of interest by moving a visual field of the endoscope. The processor is configured to stop the digital tracking processing when the first target region reaches the region of interest, and execute only the physical tracking processing thereafter.

### CROSS-REFERENCE TO RELATED APPLICATIONS

This is a continuation of International Application PCT/JP2022/003245 which is hereby incorporated by reference herein in its entirety.

### TECHNICAL FIELD

The present invention relates to an endoscope system, a control method, and a recording medium.

### BACKGROUND ART

In the related art, there is a known technology in endoscopic surgery, in which an endoscope camera image displayed on a display device is made to automatically track an object such as a tool (for example, see Patent Literature 1). Patent Literature 1 discloses a system using a high-resolution image, as well as digital zooming and digital panning techniques, to make the image digitally and automatically track an object without physically moving an endoscope. Specifically, a partial region, such as a region of interest, is selected from a high-resolution and wide-field full-sized image, a digitally zoomed image is generated from the selected region, and the digitally zoomed image is displayed on a display device. As a result of moving a region to be selected in the full-sized image, the digitally zoomed image digitally tracks an object.

### CITATION LIST

### Patent Literature

{PTL 1} U.S. Pat. No. 10,038,888, Specification

### SUMMARY OF INVENTION

An aspect of the present invention is an endoscope system including: an endoscope that acquires a captured image; a drive mechanism that moves a visual field of the endoscope by moving at least a distal end portion of the endoscope; and a processor configured to control an observation image displayed on a display and the drive mechanism, the observation image being an image generated from a portion of the captured image. The processor is configured to: detect a prescribed region of interest in the captured image; select the portion of the captured image as a display area and generate the observation image from the display area; and cooperatively execute digital tracking processing and physical tracking processing, thereby making the observation image track the region of interest. The digital tracking processing is processing in which a prescribed first target region at a center of the display area or in a vicinity of the center of the display area is made to track the region of interest by changing a position of the display area in the captured image. The physical tracking processing is processing in which a prescribed second target region at a center of the captured image or in a vicinity of the center of the captured image is made to track the region of interest by moving the visual field of the endoscope. The processor is configured to stop the digital tracking processing when the first target region reaches the region of interest, and execute only the physical tracking processing thereafter.

Another aspect of the present invention is a control method for controlling an observation image displayed on a display and movement of a visual field of an endoscope, the observation image being an image generated from a portion of a captured image acquired by the endoscope, the control method including: detecting a prescribed region of interest in the captured image; selecting the portion of the captured image as a display area and generating the observation image from the display area; and cooperatively executing digital tracking processing and physical tracking processing, thereby making the observation image track the region of interest. The digital tracking processing is processing in which a prescribed first target region at a center of the display area or in a vicinity of the center of the display area is made to track the region of interest by changing a position of the display area in the captured image. The physical tracking processing is processing in which a prescribed second target region at a center of the captured image or in a vicinity of the center of the captured image is made to track the region of interest by moving the visual field of the endoscope. The cooperatively executing the digital tracking processing and the physical tracking processing includes stopping the digital tracking processing when the first target region reaches the region of interest, and executing only the physical tracking processing thereafter.

Another aspect of the present invention is a non-transitory computer-readable recording medium having a control program for controlling an observation image displayed on a display and movement of a visual field of an endoscope, the observation image being an image generated from a portion of a captured image acquired by the endoscope stored therein, the program causing a computer to execute functions of: detecting a prescribed region of interest in the captured image; selecting the portion of the captured image as a display area and generating the observation image from the display area; and cooperatively executing digital tracking processing and physical tracking processing, thereby making the observation image track the region of interest. The digital tracking processing is processing in which a prescribed first target region at a center of the display area or in a vicinity of the center of the display area is made to track the region of interest by changing a position of the display area in the captured image. The physical tracking processing is processing in which a prescribed second target region at a center of the captured image or in a vicinity of the center of the captured image is made to track the region of interest by moving the visual field of the endoscope. The cooperatively executing the digital tracking processing and the physical tracking processing includes stopping the digital tracking processing when the first target region reaches the region of interest, and executing only the physical tracking processing thereafter.

Another aspect of the present invention is a non-transitory computer-readable recording medium having a control program for controlling an observation image displayed on a display and movement of a visual field of an endoscope, the observation image being an image generated from a portion of a captured image acquired by the endoscope stored therein, the program causing a computer to execute functions of: detecting a prescribed region of interest in the captured image; selecting the portion of the captured image as a display area and generating the observation image from the display area; and cooperatively executing digital tracking processing and physical tracking processing, thereby making the observation image track the region of interest. The digital tracking processing is processing in which a prescribed first target region at a center of the display area or in a vicinity of the center of the display area is made to track the region of interest by changing a position of the display area in the captured image. The physical tracking processing is processing in which a prescribed second target region at a center of the captured image or in a vicinity of the center of the captured image is made to track the region of interest by moving the visual field of the endoscope. The cooperatively executing the digital tracking processing and the physical tracking processing includes stopping the digital tracking processing when the first target region reaches the region of interest, and executing only the physical tracking processing thereafter.

### BRIEF DESCRIPTION OF DRAWINGS

FIG. 1 is an overall configuration diagram of an example of an endoscope system according to an embodiment of the present invention.

FIG. 2 is a block diagram showing the overall configuration of the endoscope system in FIG. 1.

FIG. 3 is a diagram for explaining movement of a visual field due to bending of a bending portion of an endoscope.

FIG. 4A is a diagram for explaining processing for making an observation image track a ROI and is a diagram showing an example of a captured image and a display area.

FIG. 4B is a diagram for explaining the processing for making the observation image track the ROI and is a diagram showing an example of the captured image and the display area.

FIG. 4C is a diagram for explaining the processing for making the observation image track the ROI and is a diagram showing an example of the captured image and the display area.

FIG. 4D is a diagram for explaining the processing for making the observation image track the ROI and is a diagram showing an example of the captured image and the display area.

FIG. 4E is a diagram for explaining the processing for making the observation image track the ROI and is a diagram showing an example of the captured image and the display area.

FIG. 5A is a flowchart of a control method according to an embodiment of the present invention.

FIG. 5B is a flowchart of a digital tracking processing routine in FIG. 5A.

FIG. 5C is a flowchart of a physical tracking processing routine in FIG. 5.

FIG. 6A is a diagram showing an example of temporal changes in the movement amount of the display area and the movement amount of the visual field.

FIG. 6B is a diagram showing another example of the temporal changes in the movement amount of the display area and the movement amount of the visual field.

FIG. 6C is a diagram showing another example of the temporal changes in the movement amount of the display area and the movement amount of the visual field.

FIG. 7 is a diagram for explaining a movable range of the display area in the captured image.

FIG. 8A is a diagram showing an example of the temporal changes in the movement amount of the display area and the movement amount of the visual field in a case in which the physical tracking processing is started before the digital tracking processing.

FIG. 8B is a diagram showing an example of the temporal changes in the movement amount of the display area and the movement amount of the visual field in a case in which the digital tracking processing is started before the physical tracking processing.

FIG. 9A is a diagram showing an example of the temporal changes in the movement amount of the display area and the movement amount of the visual field in a case in which a dead zone is set in a central portion of the captured image.

FIG. 9B is a diagram showing an example of the temporal changes in the movement amount of the display area and the movement amount of the visual field in a case in which, in the physical tracking processing, the visual field is moved until the ROI passes across a second target region in the captured image.

FIG. 10A is a diagram showing an example of the captured image and the display area in the tracking processing in FIG. 9A.

FIG. 10B is a diagram showing an example of the captured image and the display area in the tracking processing in FIG. 9B.

### DESCRIPTION OF EMBODIMENT

An endoscope system, a control method, and a recording medium according to an embodiment of the present invention will be described below with reference to the drawings.

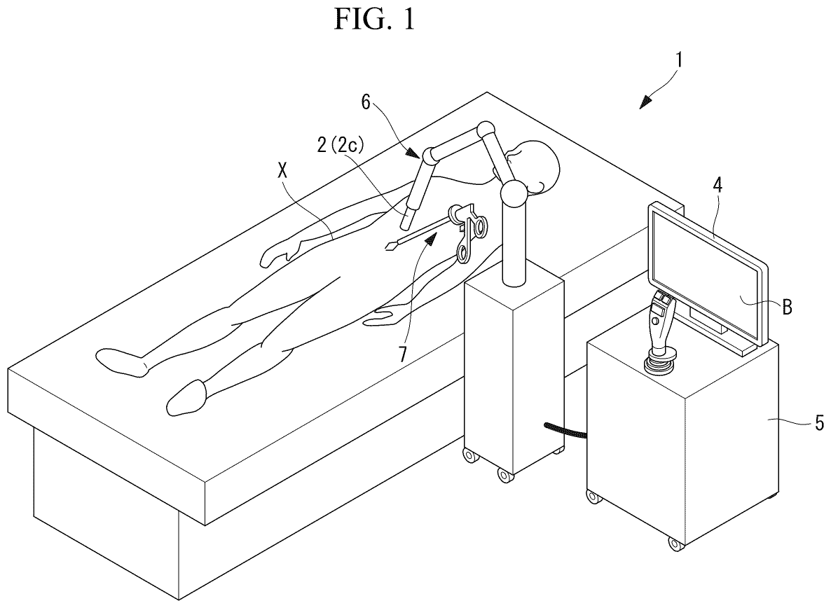

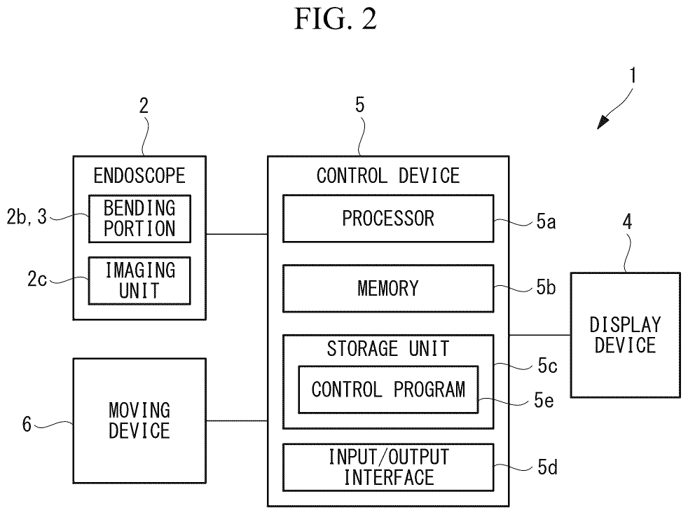

As shown in FIGS. 1 and 2, an endoscope system 1 according to this embodiment includes: an endoscope 2 that is inserted into the body; a drive mechanism 3 that moves at least a distal end portion of the endoscope 2; a display device 4; and a control device 5 that controls the drive mechanism 3 and an image displayed on the display device 4.

FIG. 1 shows, as an example, the endoscope system 1 for laparoscopic surgery in which an affected site is treated by means of a treatment tool 7 that is inserted into the abdominal cavity of a patient X, while the treatment tool 7 is observed by means of the endoscope 2.

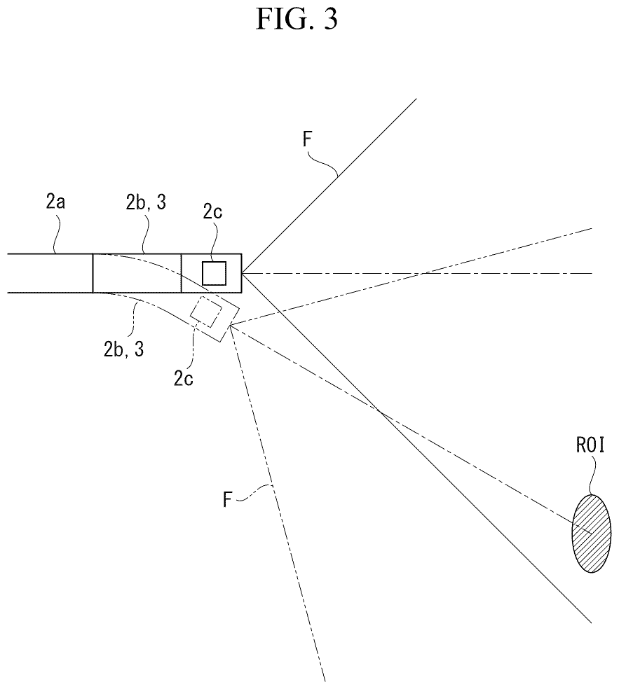

As shown in FIG. 3, the endoscope 2 has: a rigid elongated insertion portion 2a; an electrically driven bending portion 2b that is provided in the insertion portion 2a; and an imaging unit 2c that is provided in a distal end portion of the insertion portion 2a. The drive mechanism 3 is the bending portion 2b. The bending portion 2b can be bent in a direction intersecting a longitudinal axis of the insertion portion 2a, and the distal end portion of the insertion portion 2a and a visual field F of the endoscope 2 move due to bending of the bending portion 2b. In FIG. 3, a one-dot chain line indicates an optical axis of the endoscope 2. The endoscope 2 is, for example, inserted into the body through a trocar penetrating the body wall and is supported by the trocar.

The endoscope system 1 may further include a moving device 6 for holding and moving the endoscope 2. The moving device 6 includes, for example, an electric holder configured from an articulated robot arm and is controlled by the control device 5. In this case, the drive mechanism 3 may be the moving device 6 instead of the bending portion 2b, and the endoscope 2 need not have the bending portion 2b. Alternatively, both the bending portion 2b and the moving device 6 may be used as the drive mechanism 3 to move the visual field F of the endoscope 2.

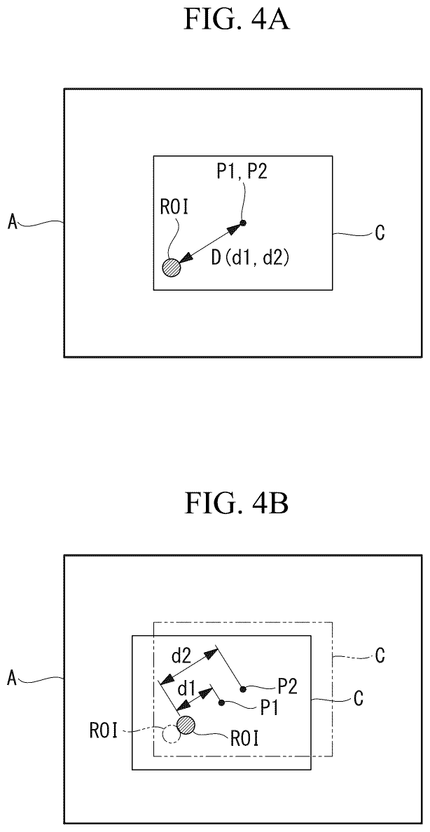

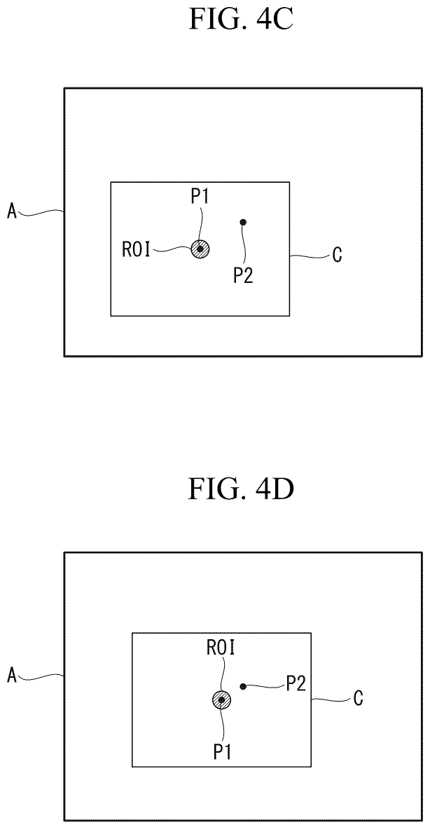

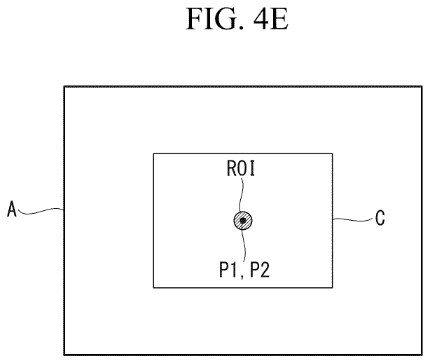

The imaging unit 2c has an imaging element, such as a CCD image sensor or a CMOS image sensor, and acquires a captured image A (see FIGS. 4A to 4E) including a prescribed region of interest (ROI). The captured image A is transmitted from the endoscope 2 to the control device 5, an observation image B is generated from the captured image A in the control device 5, and the observation image B is displayed on the display device 4. As will be described later, the observation image B is a digitally zoomed image of a portion of the captured image A. Therefore, it is preferable that the captured image A be a wide-field and high-resolution image, and it is preferable that a wide-angle endoscope 2 and a high-resolution imaging unit 2c be used. The display device 4 is an arbitrary display, such as a liquid crystal display or an organic EL display.

The control device 5 controls the operation of the bending portion 2b and/or the moving device 6 serving as the drive mechanism 3 and the observation image B displayed on the display device 4. Specifically, as shown in FIG. 2, the control device 5 includes at least one processor 5a, a memory 5b, a storage unit 5c, and an input/output interface 5d.

The control device 5 is connected to the other peripheral devices 2, 3, 4, 6 via the input/output interface 5d, and receives and transmits images, signals, etc. via the input/output interface 5d.

The memory 5b is, for example, a semiconductor memory including a read-only memory (ROM) or random access memory (RAM) area.

The storage unit 5c is a non-transitory computer-readable recording medium and is, for example, a non-volatile recording medium including a hard disk or a semiconductor memory such as a flash memory. The storage unit 5c stores various programs including a control program 5e, and data required for processing by the processor 5a. Part of the processing executed by the processor 5a, which will be described later, may be implemented by a dedicated logic circuit, such as a field programmable gate array (FPGA), a system-on-a-chip (SoC), an application specific integrated circuit (ASIC), or a programmable logic device (PLD), hardware, or the like.

As shown in FIGS. 4A to 4E, the processor 5a selects a portion in the captured image A as a display area C, enlarges the display area C by means of digital processing to generate an observation image B, and causes the observation image B to be displayed on the display device 4. The display area C is, for example, a rectangular area having a prescribed size.

Here, the ROI is a region to which an operator pays close attention during surgery and is, for example, the treatment tool 7, tissue, or an organ. For example, when the operator moves the treatment tool 7, or when tissue or an organ deforms in accordance with the progress of the surgery, the ROI moves in the body. When the ROI moves away from a prescribed target region P2 (described later) in the captured image A, for example, when the distance from the target region P2 to the ROI in the captured image A exceeds a prescribed threshold, the processor 5a controls the position of the display area C in the captured image A and the drive mechanism 3, thereby executing a control method for automatically adjusting the observation image B displayed on the display device 4 to the position of the ROI.

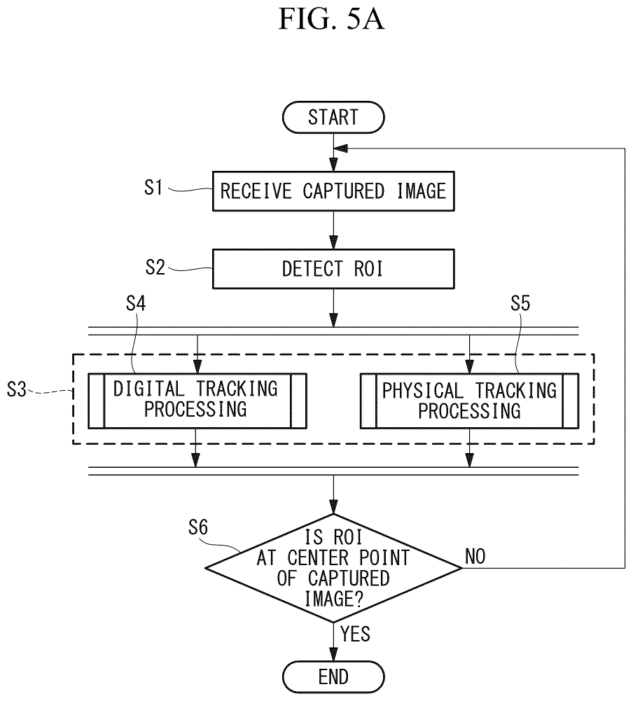

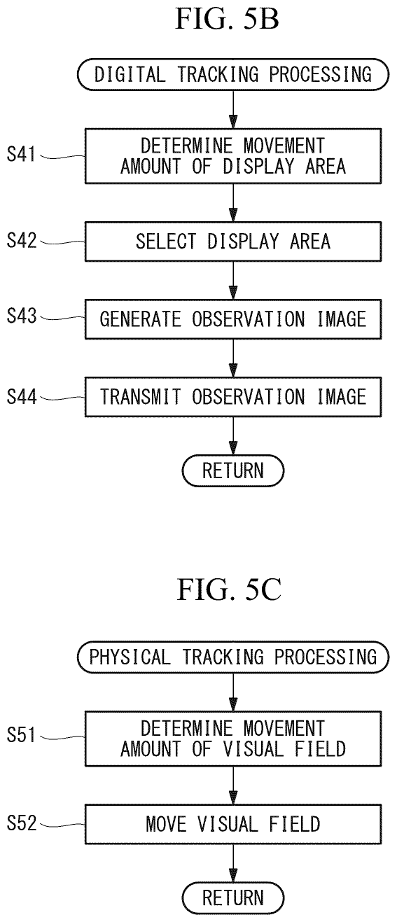

Next, the control method executed by the processor 5a will be described with reference to FIGS. 5A to 5C. The processor 5a executes the control method by executing processing according to the control program 5e read from the storage unit 5c into the memory 5b.

As shown in FIG. 5A, the control method according to this embodiment includes: step S1 of receiving a captured image A; step S2 of detecting a prescribed ROI in the captured image A; and step S3 of making an observation image B track the ROI.

FIGS. 4A to 4E show the captured images A acquired by the endoscope 2 during the execution of the control method. Specifically, FIG. 4A shows the captured image A at the start of the control method, FIG. 4E shows the captured image A at the end of the control method, and time proceeds from FIG. 4A to FIG. 4E. As shown in FIG. 4A, at the start of the control method, the display area C is normally disposed in a central portion of the captured image A where a first target region P1 coincides with a second target region P2. The first target region P1 is a prescribed region at the center or in the vicinity of the center of the display area C, and the second target region P2 is a prescribed region at the center or in the vicinity of the center of the captured image A. Each of the target regions P1, P2 may be a single point or a two-dimensional region having a certain area.

In step S2, the processor 5a detects the ROI in the captured image A by using a publicly known means such as, for example, image recognition with artificial intelligence or detection of a marker provided in advance in the ROI.

Step S3 includes step S4 of performing digital tracking processing and step S5 of performing physical tracking processing.

The digital tracking processing is processing in which a prescribed first target region P1 in the display area C is made to digitally track the ROI by changing the position of the display area C in the captured image A.

The physical tracking processing is processing in which a prescribed second target region P2 in the captured image A is made to physically (mechanically) track the ROI by operating the drive mechanism 3 so as to move the visual field F of the endoscope 2 at a constant speed.

The processor 5a simultaneously starts the digital tracking processing and the physical tracking processing, and simultaneously executes the digital tracking processing and the physical tracking processing in a cooperative manner.

As shown in FIG. 5B, step S4 includes: step S41 of determining a movement amount d1 of the display area C; step S42 of selecting the display area C from the captured image A on the basis of the movement amount d1; step S43 of generating an observation image B from the display area C; and step S44 of transmitting the observation image B to the display device 4.

As shown in FIGS. 4A and 4B, in step S41, the processor 5a determines the movement amount d1 of the display area C on the basis of the positional relationship between the first target region P1 in the display area C and the ROI. The movement amount d1 is a movement amount of the display area C required for moving the first target region P1 to the ROI.

Next, as shown in FIGS. 4B and 4C, in step S42, the processor 5a changes the position of the display area C from the current position in a direction in which the movement amount d1 approaches zero, in other words, in a direction in which the first target region P1 approaches the ROI. The movement amount of the display area C at this time is set, for example, such that the moving speed of an imaging object in the observation image B on the display device 4 is an appropriate speed for a user. In FIG. 4B, a two-dot chain line indicates the display area C before the position thereof is changed.

Next, in step S43, the processor 5a selects the display area C at the changed position from the captured image A, and enlarges the size of the selected display area C to generate an observation image B.

Next, in step S44, the processor 5a transmits the generated observation image B to the display device 4, and causes the observation image B to be displayed on the display device 4.

As a result of the digital tracking processing in step S4, the display area C moves to a position deviated from the center of the captured image A. The physical tracking processing is processing for returning the display area C to the center of the captured image A.

As shown in FIG. 5C, step S5 includes step S51 of determining a movement amount d2 of the visual field F and step S52 of moving the visual field F.

As shown in FIGS. 4A and 4B, in step S51, the processor 5a determines the movement amount d2 of the visual field F on the basis of the positional relationship between the second target region P2 in the captured image A and the ROI. The movement amount d2 is a movement amount of the visual field F required for moving the second target region P2 to the ROI.

Next, as shown in FIG. 4B, in step S52, the processor 5a causes the visual field F to move from the current position in a direction in which the movement amount d2 approaches zero, in other words, in a direction in which the second target region P2 approaches the ROI. At this time, the visual field F is moved, for example, at a maximum speed that could be achieved by the drive mechanism 3. In FIG. 4B, a two-dot chain line indicates the ROI before the visual field F is moved.

As a result of the digital tracking processing S4 and the physical tracking processing S5 being simultaneously executed once, the ROI in the observation image B on the display device 4 moves toward the center of the observation image B by a total movement amount, which is the sum of the movement amount of the display area C and the movement amount of the visual field F.

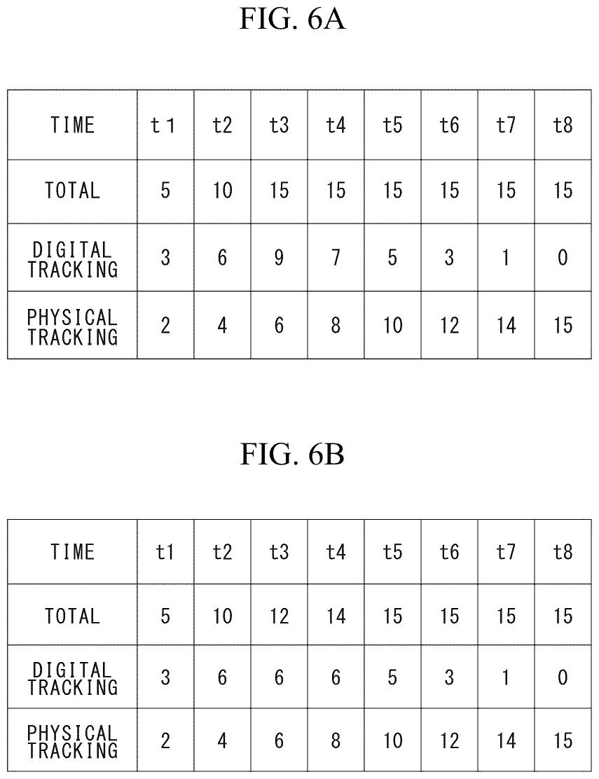

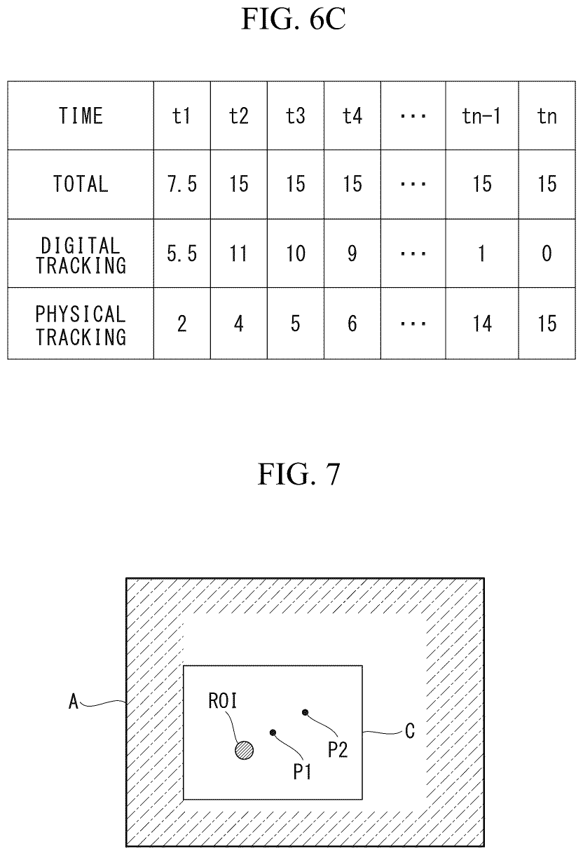

As shown in FIGS. 4A to 4E and FIG. 6A, as a result of the processor 5a repeatedly executing steps S1 to S5, the respective movement amounts d1, d2 gradually approach zero.

The processor 5a repeats steps S1 to S5 until the second target region P2 in the captured image A reaches the ROI (YES in step S6). In an example in FIG. 6A, a distance D (see FIG. 4A) from the second target region P2 to the ROI at the start of tracking is “15”, the movement amount per unit time in the digital tracking processing (movement amount from time ti to time ti+1) is “3”, the movement amount per unit time in the physical tracking processing (movement amount from time ti to time ti+1) is “2”, and step S3 is repeated until time t8.

Note that, in FIGS. 6A to 6C and FIGS. 8A to 9B, “digital tracking” indicates the movement amount of the display area C due to the digital tracking processing, “physical tracking” indicates the movement amount of the visual field F due to the physical tracking processing, and “total” indicates the sum of the movement amount of the display area C and the movement amount of the visual field F. In addition, the movement amounts each indicate the total movement amount from the position at the start of tracking, and also indicate a two-dimensional movement amount in an image plane in the coordinate system of the captured image A. In addition, time t1, t2, t3, . . . (s) proceeds from the left to the right in these figures.

Here, because the digital tracking processing and the physical tracking processing are simultaneously executed by means of parallel processing, as shown in FIG. 4C, the first target region P1 in the display area C reaches the ROI before the second target region P2 in the captured image A reaches the ROI, and the ROI is disposed at the center or in the vicinity of the center of the observation image B on the display device 4. In the example in FIG. 6A, the first target region P1 reaches the ROI at time t3.

The processor 5a stops the digital tracking processing when the first target region P1 reaches the ROI and the movement amount d1 becomes zero, and subsequently, as shown in FIGS. 4D and 4E, executes only the physical tracking processing until the second target region P2 reaches the ROI and the movement amount d2 becomes zero.

In other words, after the first target region P1 reaches the ROI, the processor 5a causes the visual field F to move in the direction in which the second target region P2 approaches the ROI, and at the same time, changes the position of the display area C in the captured image A by a movement amount equal to the movement amount of the visual field F, in a direction in which the first target region P1 approaches the second target region P2. By doing so, the processor 5a moves the display area C to the center of the captured image A while maintaining the first target region P1 in the display area C at the ROI.

The movement of the ROI, such as the treatment tool 7, may be faster than the physical (mechanical) movement of the distal end portion of the endoscope 2. Therefore, in a case in which the captured image A or the observation image B is made to physically track the ROI only by moving the visual field F, there is a problem in that the tracking responsiveness is low. Meanwhile, in the case in which the observation image B is made to digitally track the ROI by changing the position of the display area C in the captured image A, it is possible to easily realize a high tracking responsiveness.

With this embodiment, there is an advantage in that it is possible to realize a high tracking responsiveness by combining the physical tracking processing and the digital tracking processing.

In particular, because the moving speed of the visual field F due to the operation of the drive mechanism 3, such as the bending portion 2b or the moving device 6, is lower than the moving speed of the ROI, in the initial stage of tracking, it is difficult to capture the ROI at the center of the captured image A only by moving the visual field F. By starting the digital tracking processing S4 at the same time as the start of tracking, there is an advantage in that it is possible to realize a high responsiveness even in the initial stage of tracking.

In addition, in general, the image quality is good in a central portion of an image and deteriorates in a peripheral edge portion of the image. In particular, in the case of the wide-angle endoscope 2, distortion occurs in the peripheral edge portion of the captured image A. With this embodiment, after the second target region P2 in the display area C reaches the ROI at a position away from the central portion of the captured image A, the display area C quickly moves to the central portion of the captured image A due to the movement of the visual field F, and the image quality of the observation image B is quickly improved. Therefore, there is an advantage in that, even if the image quality of the observation image B is temporarily deteriorated due to the digital tracking processing, it is possible to quickly provide the user with the observation image B having a good image quality with no or less distortion.

In addition, in a case in which only the bending portion 2b is used as the drive mechanism 3 for moving the visual field F, the visual field F moves only due to the movement of the distal end portion of the endoscope 2 disposed in the body. Therefore, the moving device 6, such as a robot arm, that holds the endoscope 2 does not move, and thus, it is possible to prevent interference between the moving device 6 and the operator.

It is also possible to move the visual field F by pivoting the entire endoscope 2 about a prescribed pivot point or by moving the entire endoscope 2 by means of the moving device 6. In this case, there is a possibility that the moving endoscope 2 or moving device 6 interferes with the operator etc. in the surrounding area.

Meanwhile, in a case in which the observation direction of an imaging object, such as biological tissue, needs to be maintained, it is preferable to use the moving device 6 as the drive mechanism 3. In other words, in a case in which the visual field F is moved due to bending of the bending portion 2b, the observation direction of the imaging object changes. In contrast, in the case in which the endoscope 2 is moved by the moving device 6, such as an electric holder, it is possible to move the visual field F while maintaining the observation direction by translationally moving the endoscope 2 while maintaining the orientation of the distal end of the electric holder.

Although the movable range of the display area C in the digital tracking processing may be the entire area of the captured image A in the abovementioned embodiment, alternatively, the movement of the display area C in the digital tracking processing may be restricted within a prescribed area of the captured image A.

As described above, the image quality of the captured image A may deteriorate in the peripheral edge portion thereof. Therefore, for example, as shown in FIG. 6B, an upper limit may be set for the total movement amount (distance from the second target region P2 to the first target region P1) of the display area C, and the display area C may be movable in the captured image A within a range that does not exceed the upper limit. In an example in FIG. 6B, the upper limit of the total movement amount of the display area C is “6”.

With this configuration, as shown in FIG. 7, a central region excluding the peripheral edge portion indicated by hatching is set to be the prescribed area, and the display area C is movable only in the central region of the captured image A. Therefore, it is possible to prevent deterioration in the image quality of the observation image B displayed on the display device 4, thereby providing the user with the observation image B having a better image quality.

Although the moving speed (in other words, the movement amount from time ti to time ti+1) of the visual field F in the physical tracking processing is set to be constant in the abovementioned embodiment, alternatively, the moving speed of the visual field F may change.

For example, as shown in FIG. 6C, the processor 5a causes the visual field F to move at the maximum speed until the first target region P1 in the display area C reaches the ROI in order to increase the tracking responsiveness with respect to the movement of the ROI. Then, after the first target region P1 reaches the ROI, the processor 5a may reduce the moving speed of the visual field F to a speed lower than the maximum speed. In an example in FIG. 6C, the moving speed of the visual field F is “2” until time t2 and is “1” at time t3 and thereafter.

When the moving speed of the visual field F is high, there are cases in which a user is bothered with an abrupt change in the image quality of the observation image B on the display device 4. By reducing the moving speed of the visual field F after the first target region P1 reaches the ROI, it is possible to slowly change the image quality of the observation image B so that the image quality change does not bother the user.

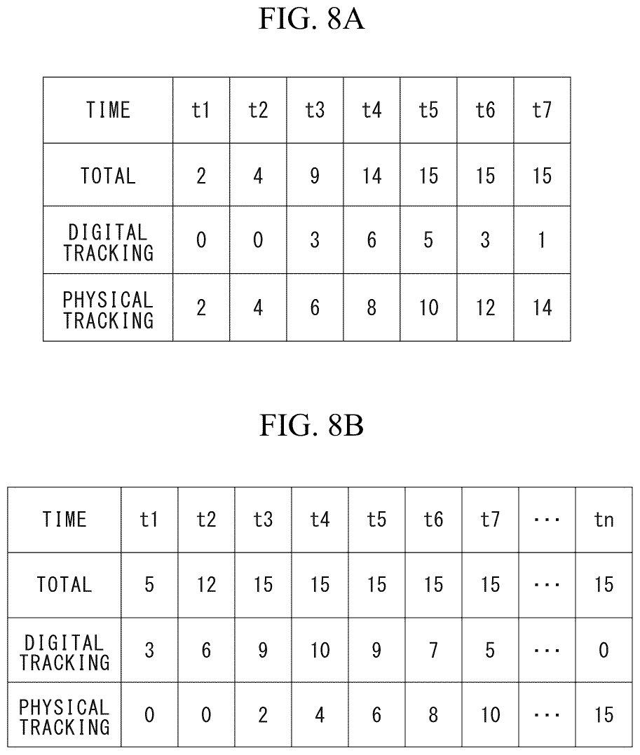

Although the processor 5a simultaneously starts the digital tracking processing S4 and the physical tracking processing S5 in the abovementioned embodiment, alternatively, as shown in FIGS. 8A and 8B, the digital tracking processing S4 and the physical tracking processing S5 may be started at different timing.

FIG. 8A shows an example of the movement amounts in a case in which the processor 5a starts the physical tracking processing S5 and subsequently starts the digital tracking processing S4. While the digital tracking processing can increase the tracking speed, said processing may cause an abrupt change in the image quality, such as distortion. By starting the digital tracking processing S4 after the physical tracking processing S5 is started and the second target region P2 approaches the ROI to some extent, it is possible to prevent an abrupt change in the image quality.

FIG. 8B shows an example of the movement amounts in a case in which the processor 5a starts the digital tracking processing S4 and subsequently starts the physical tracking processing S5. With this configuration, it is possible to reduce the burden on hardware such as the drive mechanism 3.

Although the processor 5a causes the visual field F to move in the physical tracking processing S5 until the ROI reaches the second target region P2 in the captured image A in the abovementioned embodiment, alternatively, the visual field F may be moved until the ROI reaches another position in the vicinity of the second target region P2. In other words, the position in the captured image A where the ROI should finally reach can be changed, as appropriate, according to surgical scenes, user requirements, etc.

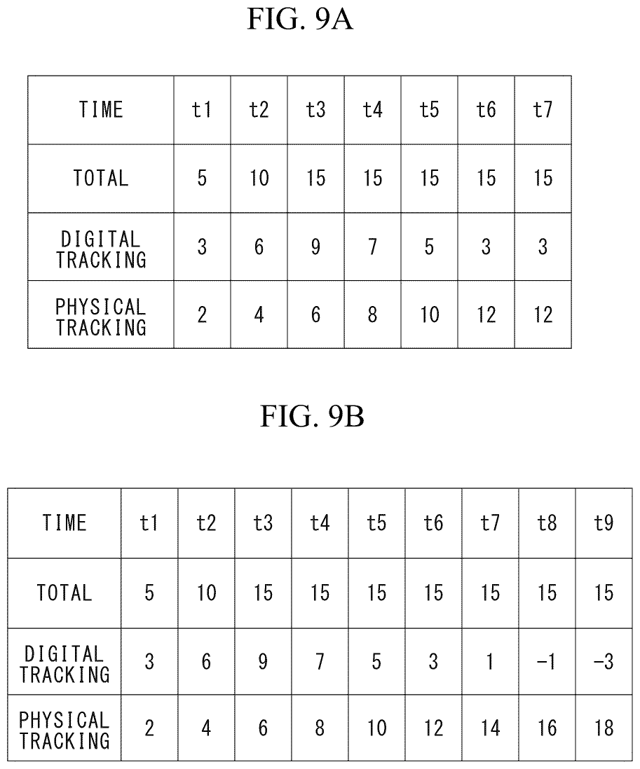

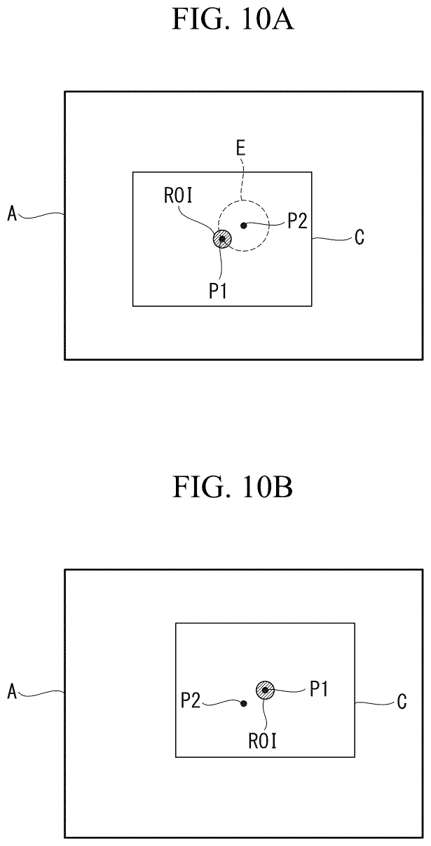

For example, as shown in FIGS. 9A and 10A, a dead zone E including the second target region P2 may be set at the center of the captured image A, and the processor 5a may end the physical tracking processing S5 when the ROI disposed outside the dead zone E enters the dead zone E. In this case, the processor 5a causes the visual field F to move until the ROI reaches a position just before the second target region P2. Alternatively, as shown in FIGS. 9B and 10B, the processor 5a may cause the visual field F to move until the ROI passes across the second target region P2. For example, the processor 5a may end the physical tracking processing S5 when the ROI passes across the second target region P2 by a prescribed distance.

In the case of the example in FIGS. 9A and 10A, a larger space is formed on the upper left side of the ROI after the tracking processing. Therefore, in the next tracking processing, the display area C can be made to perform tracking on the upper left side with a sufficient margin. Meanwhile, in the case of the example in FIGS. 9B and 10B, a larger space is formed on the lower right side of the ROI after the tracking processing. Therefore, in the next tracking processing, the display area C can be made to perform tracking on the lower right side with a sufficient margin.

The examples in FIGS. 9A to 10B are suitable, for example, in a case in which the next movement direction of the ROI can be predicted in advance. In other words, the position where the ROI should finally reach may be determined so that a large space is formed on the side where the ROI moves next.

In the abovementioned embodiment, the first target region P1 is set to be a point at the center or in the vicinity of the center of the display area C so that the user can easily observe the ROI in the observation image B; however, the position of the first target region P1 may be an arbitrary position other than the center or the vicinity of the center of the display area C. For example, the position of the first target region P1 can be changed, as appropriate, according to requirements from a user such as an operator.

In addition, in the abovementioned embodiment, the second target region P2 is set to be a point at the center or in the vicinity of the center of the captured image A in order to realize a good image quality in the observation image B even when the wide-angle endoscope 2 is used; however, the position of the second target region P2 may be an arbitrary position other than the center or the vicinity of the center of the captured image A. For example, the position of the second target region P2 can be changed, as appropriate, in a case in which the image quality is good over the entire captured image A, or in a case in which deterioration in the image quality of the observation image B is not an issue to the user.

### REFERENCE SIGNS LIST

1 endoscope system
2 endoscope
2b bending portion, drive mechanism
3 drive mechanism
4 display device
5 control device
5a processor
6 moving device, drive mechanism
A captured image
B observation image
C display area
E dead zone
F visual field
P1 first target region
P2 second target region
ROI region of interest

1. An endoscope system comprising:
an endoscope that acquires a captured image;
a drive mechanism that moves a visual field of the endoscope by moving at least a distal end portion of the endoscope; and
a processor configured to control an observation image displayed on a display and the drive mechanism, the observation image being an image generated from a portion of the captured image,
wherein the processor is configured to:
detect a prescribed region of interest in the captured image;
select the portion of the captured image as a display area and generate the observation image from the display area; and
cooperatively execute digital tracking processing and physical tracking processing, thereby making the observation image track the region of interest,
wherein the digital tracking processing is processing in which a prescribed first target region at a center of the display area or in a vicinity of the center of the display area is made to track the region of interest by changing a position of the display area in the captured image,
the physical tracking processing is processing in which a prescribed second target region at a center of the captured image or in a vicinity of the center of the captured image is made to track the region of interest by moving the visual field of the endoscope, and
the processor is configured to stop the digital tracking processing when the first target region reaches the region of interest, and execute only the physical tracking processing thereafter.

2. The endoscope system according to claim 1, wherein the processor is configured to execute the digital tracking processing and the physical tracking processing by means of parallel processing.

3. The endoscope system according to claim 2, wherein the processor is configured to start the digital tracking processing and subsequently start the physical tracking processing.

4. The endoscope system according to claim 2, wherein the processor is configured to start the physical tracking processing and subsequently start the digital tracking processing.

5. The endoscope system according to claim 1, wherein, in the digital tracking processing, movement of the display area is restricted within a prescribed area of the captured image.

6. The endoscope system according to claim 1, wherein, after the first target region reaches the region of interest, the processor is configured to reduce a moving speed of the visual field in the physical tracking processing.

7. The endoscope system according to claim 1, wherein:
a dead zone including the second target region is set in a central portion of the captured image; and
the processor is configured to end the physical tracking processing when the region of interest enters the dead zone.

8. The endoscope system according to claim 1, wherein, in the physical tracking processing, the processor is configured to cause the visual field to move until the region of interest passes across the second target region.

9. The endoscope system according to claim 1, wherein the drive mechanism includes a bending portion provided in the distal end portion of the endoscope.

10. The endoscope system according to claim 1, wherein the drive mechanism includes a moving device that holds and moves the endoscope.

11. A control method for controlling an observation image displayed on a display and movement of a visual field of an endoscope, the observation image being an image generated from a portion of a captured image acquired by the endoscope, the control method comprising:
detecting a prescribed region of interest in the captured image;
selecting the portion of the captured image as a display area and generating the observation image from the display area; and
cooperatively executing digital tracking processing and physical tracking processing, thereby making the observation image track the region of interest,
wherein the digital tracking processing is processing in which a prescribed first target region at a center of the display area or in a vicinity of the center of the display area is made to track the region of interest by changing a position of the display area in the captured image,
the physical tracking processing is processing in which a prescribed second target region at a center of the captured image or in a vicinity of the center of the captured image is made to track the region of interest by moving the visual field of the endoscope, and
the cooperatively executing the digital tracking processing and the physical tracking processing comprises stopping the digital tracking processing when the first target region reaches the region of interest, and executing only the physical tracking processing thereafter.

12. A non-transitory computer-readable recording medium having a control program for controlling an observation image displayed on a display and movement of a visual field of an endoscope, the observation image being an image generated from a portion of a captured image acquired by the endoscope stored therein, the program causing a computer to execute functions of:
detecting a prescribed region of interest in the captured image;
selecting the portion of the captured image as a display area and generating the observation image from the display area; and
cooperatively executing digital tracking processing and physical tracking processing, thereby making the observation image track the region of interest,
wherein the digital tracking processing is processing in which a prescribed first target region at a center of the display area or in a vicinity of the center of the display area is made to track the region of interest by changing a position of the display area in the captured image,
the physical tracking processing is processing in which a prescribed second target region at a center of the captured image or in a vicinity of the center of the captured image is made to track the region of interest by moving the visual field of the endoscope, and
the cooperatively executing the digital tracking processing and the physical tracking processing comprises stopping the digital tracking processing when the first target region reaches the region of interest, and executing only the physical tracking processing thereafter.
